(19) United States
(12) Patent Application Publication
(10) Pub. No.: US 2017/0074769 A1
(43) Pub. Date: Mar. 16, 2017

(54) SPECIMEN EVALUATION METHOD

(71) Applicant: OLYMPUS CORPORATION, Tokyo (JP)

(72) Inventor: Kazutaka NISHIKAWA, Tokyo (JP)

(73) Assignee: OLYMPUS CORPORATION, Tokyo (JP)

(21) Appl. No.: 15/359,930

(22) Filed: Nov. 23, 2016

Related U.S. Application Data
(63) Continuation of application No. PCT/JP2015/054724, filed on Feb. 20, 2015.

(30) Foreign Application Priority Data
Jun. 11, 2014 (JP) 2014-120575

(51) Int. Cl. G01N 15/04; G01N 33/483

(52) U.S. Cl. CPC G01N 15/04; G01N 33/4833

(57) ABSTRACT

A specimen evaluation method including: a stirring step of stirring a measurement solution that contains a specimen; a measurement step of measuring, after the stirring step, the density of cells at a predetermined depth position in the measurement solution; a remeasurement step of measuring again, after the measurement step, the density of the cells at the predetermined depth position in the measurement solution; a change calculation step of calculating the difference between the density of the cells measured in the measurement step and the density of the cells measured in the remeasurement step; and evaluation steps of evaluating whether the specimen is good or not on the basis of the difference in the density of the cells.

### CROSS-REFERENCE TO RELATED APPLICATIONS

This is a continuation of International Application PCT/JP2015/054724 which is hereby incorporated by reference herein in its entirety.

This application is based on Japanese Patent Application No. 2014-120575, the contents of which are incorporated herein by reference.

### TECHNICAL FIELD

The present invention relates to a specimen evaluation method and, in particular, to a method for evaluating whether a specimen collected in a biopsy is appropriate for examination, on the basis of the number of cells contained in the specimen.

### BACKGROUND ART

In a biopsy, it is important to reliably collect a sufficient amount of a specimen for examination. If the amount of the collected specimen is not sufficient, an accurate examination result cannot be obtained, and re-collection and re-examination of the specimen are needed. However, when an object to be examined is a micrometer-scale cell, it is difficult to confirm only visually whether the amount of the specimen is sufficient or not. Thus, proposed is a method for confirming the number of cells in a collected specimen by measuring the turbidity of a fluid in which the specimen is suspended (for example, see PTL 1).

### CITATION LIST

### Patent Literature

{PTL 1} Japanese Translation of PCT International Application, Publication No. 2012-529048

### SUMMARY OF INVENTION

### Solution to Problem

The present invention provides a specimen evaluation method including: a stirring step of stirring a measurement solution that contains a specimen collected from a living body, thereby uniformly dispersing, in the measurement solution, cells contained in the specimen; a measurement step of measuring, after completion of the stirring in the stirring step, the density of the cells at a predetermined depth position in the measurement solution at a first time point; a remeasurement step of measuring again the density of the cells at the predetermined depth position in the measurement solution at a second time point after a time interval since the first time point; a change calculation step of calculating the difference between the density of the cells measured in the measurement step and the density of the cells measured in the remeasurement step; and an evaluation step of evaluating whether the specimen is good or not on the basis of the difference in the density of the cells calculated in the change calculation step.

### BRIEF DESCRIPTION OF DRAWINGS

FIG. 1 is a flowchart showing a specimen evaluation method according to a first embodiment of the present invention.

FIG. 2 is a schematic view of a spectrophotometer used in a measurement step and a remeasurement step shown in FIG. 1.

FIG. 3 is a flowchart showing a specimen evaluation method according to a second embodiment of the present invention.

FIG. 4 is a schematic view of a bright-field optical microscope used in a measurement step and a remeasurement step shown in FIG. 3.

FIG. 5 is a graph showing the temporal changes in absorbance of a sample A and a comparative sample at wavelengths of 400, 600, and 800 nm, measured in Example 1.

FIG. 6 is a graph showing details of temporal changes in absorbance of the sample A and the comparative sample at the wavelength of 600 nm.

FIG. 7 shows optical microscope images of slide samples created from (a) the sample A and (b) the comparative sample, used in Example 1.

FIG. 8 shows bright field images of a sample A obtained in Example 2 at (a) 0 seconds and (b) 60 seconds and (c) a difference image generated from the bright field images (a) and (b).

FIG. 9 shows bright field images of a sample B obtained in Example 2 at (a) 0 seconds and (b) 60 seconds and (c) a difference image generated from the bright field images (a) and (b).

FIG. 10 shows bright field images of a sample C obtained in Example 2 at (a) 0 seconds and (b) 60 seconds and (c) a difference image generated from the bright field images (a) and (b).

FIG. 11 shows bright field images of a sample D obtained in Example 2 at (a) 0 seconds and (b) 60 seconds and (c) a difference image generated from the bright field images (a) and (b).

FIG. 12 shows bright field images of a sample E obtained in Example 2 at (a) 0 seconds and (b) 60 seconds and (c) a difference image generated from the bright field images (a) and (b).

FIG. 13 shows bright field images of a comparative sample obtained in Example 2 at (a) 0 seconds and (b) 60 seconds and (c) a difference image generated from the bright field images (a) and (b).

FIG. 14 shows bright field images of the sample A at (a) 0 seconds, (b) 10 seconds, (c) 30 seconds, and (d) 60 seconds.

FIG. 15 is a table showing the number of pixels pixel in changed regions extracted from the difference images shown in FIGS. 8 to 13.

FIG. 16 is a graph showing the relationship between the cell density (vertical axis) and Δpixel (horizontal axis), of the samples C, D, and E.

FIG. 17 shows optical microscope images of slide samples created from (a) the sample A, (b) the sample B, and (c) the comparative sample, used in Example 2.

### DESCRIPTION OF EMBODIMENTS

### First Embodiment

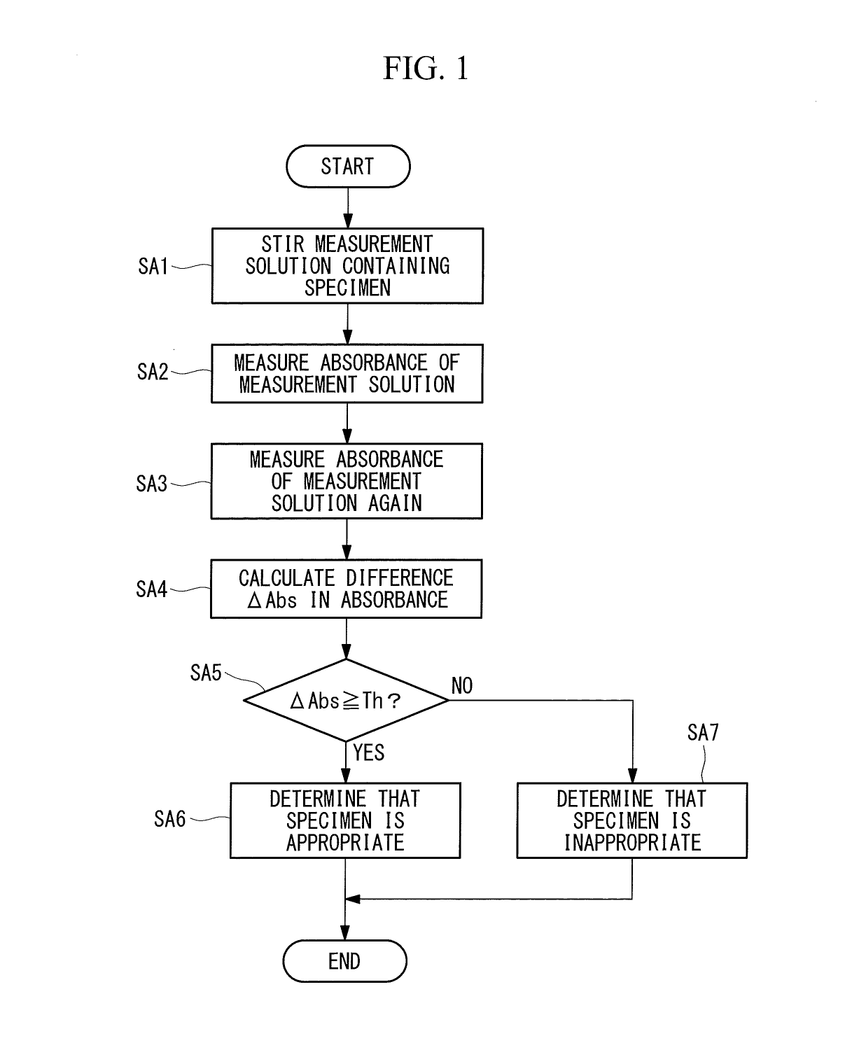

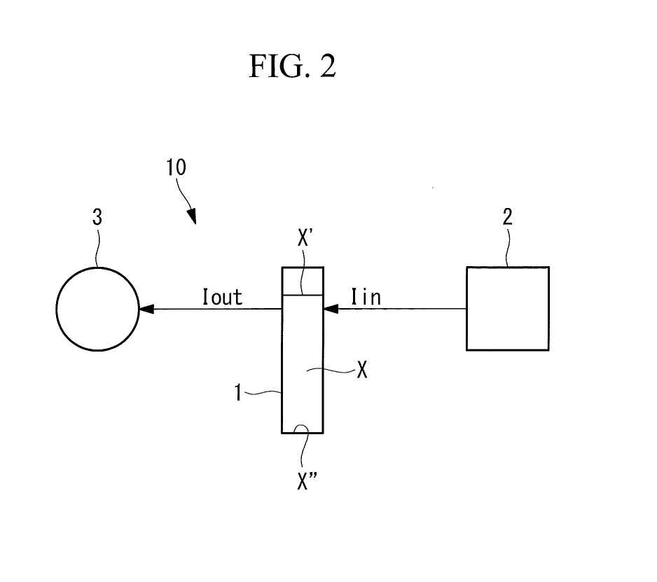

A specimen evaluation method according to a first embodiment of the present invention will be described below with reference to FIGS. 1 and 2.

As shown in FIG. 1, the specimen evaluation method of this embodiment includes: a stirring step SA1 of uniformly dispersing, in a measurement solution X, cells contained in a specimen; a measurement step SA2 of measuring the turbidity of the measurement solution X; a remeasurement step SA3 of measuring again the turbidity of the measurement solution X after a time interval since the measurement step SA2; a change calculation step SA4 of calculating the difference between the turbidity obtained in the measurement step SA2 and the turbidity obtained in the remeasurement step SA3; and evaluation steps SA5, SA6, and SA7 of evaluating whether or not the specimen is appropriate for examination on the basis of the obtained difference in turbidity.

The specimen used in this embodiment is, for example, part of tissue collected from living tissue, such as the prostate, through cell aspiration (FNA: fine-needle aspiration) using a 25-gauge puncture needle.

In the stirring step SA1, the measurement solution X to which the specimen is added is stirred, thereby uniformly dispersing the cells, which are contained in the specimen, in the measurement solution X. It is preferred that the measurement solution X be colorless and transparent, for example, a normal saline solution, so as not to affect the measurement results of absorbance measurement in the measurement step SA2 and the remeasurement step SA3.

Next, in the measurement step SA2, as shown in FIG. 2, a spectrophotometer 10 that is used in general biological research is used to measure the turbidity of the measurement solution X. The spectrophotometer 10 is provided with: a light source 2; and a light detector 3 that detects measurement light lin that is radiated from the light source 2 onto the measurement solution X, which is a measurement sample, and transmitted light lout of the measurement light lin, which has been transmitted through the measurement sample.

The turbidity of the measurement solution X is proportional to the density of cells in the measurement solution X, and the density of cells in the measurement solution X is proportional to the absorbance of the measurement solution X at a wavelength λcell, at which cells show absorbance. Therefore, by measuring the absorbance of the measurement solution X at the wavelength λcell, the turbidity of the measurement solution X and the density of cells can be obtained. The wavelength λcell of the measurement light lin falls within a range from 320 nm to 1100 nm, and more preferably, within a visual range from 400 nm to 800 nm.

Here, the measurement light lin is horizontally radiated onto a predetermined depth position from a liquid surface X′ of the measurement solution X in a measurement container 1, thereby measuring the absorbance of the measurement solution X. The predetermined depth position is set in the vicinity of the liquid surface X′ of the measurement solution X, and the absorbance of an upper layer portion in the vicinity of the liquid surface X′ of the measurement solution X is measured. As the measurement container 1, a container made of an optically transparent material, such as a cuvett generally used in spectroscopic measurement, is used.

Next, in the remeasurement step SA3, the turbidity of the measurement solution X at the same depth position is measured under the same measurement conditions as those in the measurement step SA2. Specifically, the measurement light lin having the same wavelength λcell as in the measurement step SA2 is horizontally radiated onto the same depth position in the measurement solution X in the measurement container 1 as in the measurement step SA2, thereby measuring the absorbance of the upper layer portion of the measurement solution X.

Here, in the measurement step SA2 and the remeasurement step SA3, absorbance measurement is performed in a state in which the measurement solution X is stationary. In the stationary state, because the cells in the measurement solution X settle as time proceeds, the density of the cells at the upper layer portion of the measurement solution X decreases as time proceeds, and the turbidity also decreases. Therefore, an absorbance Abs2 obtained in the remeasurement step SA3 becomes smaller than an absorbance Abs1 obtained in the measurement step SA2.

It is preferred that a first turbidity-measurement time point (first time point) T1 in the measurement step SA2 be immediately after the stirring of the measurement solution X is completed in the stirring step SA1. Furthermore, it is preferred that a second turbidity-measurement time point (second time point) T2 in the remeasurement step SA3 be within 120 seconds from the time at which the stirring of the measurement solution X is completed in the stirring step S1. Furthermore, it is preferred that the time interval between the measurement time point T1 and the measurement time point T2 be 10 seconds or longer and 120 seconds or shorter.

The next change calculation step SA4 and the evaluation steps SA5, SA6, and SA7 are performed, for example, in an information-processing device, such as a computer, connected to the spectrophotometer 10, by receiving the obtained absorbance Abs1 and absorbance Abs2 from the spectrophotometer 10 and by subjecting the received absorbance Abs1 and absorbance Abs2 to arithmetic processing.

In the change calculation step SA4, the difference between the absorbance Abs1, which is obtained in the measurement step SA2, and the absorbance Abs2, which is obtained in the remeasurement step SA3, that is, ΔAbs=|Abs|-Abs2| (hereinafter, simply referred to as ΔAbs), is calculated. The difference ΔAbs corresponds to the temporal change in the density of the cells at the upper layer portion of the measurement solution X and is proportional to the number of cells contained in the entire measurement solution X.

In the evaluation steps SA5, SA6, and SA7, ΔAbs is compared with a predetermined threshold Th (Step SA5). If ΔAbs is equal to or larger than the predetermined threshold Th (YES in Step SA5), it is determined that the number of cells necessary for examination is contained in the specimen and that this specimen is thus appropriate for examination (Step SA6). On the other hand, if ΔAbs is smaller than the predetermined threshold Th (No in Step SA5), it is determined that the number of cells contained in the specimen is not sufficient for examination and that this specimen is thus not appropriate for examination (Step SA7). The predetermined threshold Th is set on the basis of the number of cells necessary for examination for which the specimen is provided. The examination may be pathological examination, in which tissue diagnosis and cytology are performed, may be measurement of a target protein, which is performed by a colorimetric method or immunoenzymetric assay, or may be a genetic test.

Here, the settling velocity of particles in a liquid will be described.

The sum of: a gravity force in the vertically downward direction; and a buoyancy force and a resistance force, which is received by a liquid, in the vertically upward direction acts on particles in the liquid. The particles in the liquid settle out while increasing their velocity at first and settle out at a constant velocity after the gravity force and the sum of the buoyancy force and the resistance force are balanced. This constant velocity is called a terminal settling velocity, and the settling velocity of particles generally means the terminal settling velocity. A terminal settling velocity V is derived from Stokes equation (1) below.

$$\begin{matrix}( Equation 1 ) & \\ V = \frac{g ( {ρ}_{s} - ρ ) {d}^{2}}{18 μ} & ( 1 )\end{matrix}$$

V: terminal settling velocity of particles (cm/sec)
ρs: density of particles (g/cm³)
ρ: density of water (g/cm³)≈1.0 g/cm³
d: diameter of particles (cm)
g: acceleration of gravity (cm/sec²)=980.7 (cm/sec²)
82 : viscosity of water (g/cm•s)≈0.01 (g/cm•s)

As an example, when the density pₛ of particles is 1.1, the terminal settling velocity V of particles having a diameter of 100 μm is 3.3 cm/min, and the terminal settling velocity V of particles having a diameter of 10 μm is 0.33 cm/min. Specifically, larger particles settle at a higher velocity.

Although a specimen collected from a living body contains, other than cells, various biological materials, for example, blood components and tissue components, the diameters of the cells are much larger than the diameters of other biological materials. In particular, cells contained in a specimen form a large aggregate in many cases. Therefore, in the measurement solution X, the settling velocities of the other biological materials are vanishingly quite small in comparison to the settling velocity of the cells.

The absorbance Abs1, which is measured in the measurement step SA2, and the absorbance Abs2, which is measured in the remeasurement step SA3, each include, in addition to the absorbance derived from the cells, the absorbance derived from the other biological materials. However, as described above, the settling velocities of the other biological materials are quite small, and thus the absorbance derived from the other biological materials measured at the measurement time point T1 and that measured at the measurement time point T2 are the same. Therefore, in ΔAbs, the absorbance derived from the other biological materials is removed, and the temporal change in the absorbance of the net cells is detected as ΔAbs. Specifically, even when the other biological materials are contained in the measurement solution X, an accurate number of cells in the measurement solution X can be estimated by calculating ΔAbs. Accordingly, it is possible to estimate an accurate number of cells contained in a specimen and to accurately evaluate whether or not the specimen is appropriate for examination.

In this embodiment, the turbidity is measured at the upper layer portion in the vicinity of the liquid surface X′ of the measurement solution X; however, the position where the turbidity is measured is not limited thereto, and the turbidity may be measured at a desired depth position. In particular, it is preferred that the turbidity be measured at a lower layer portion in the vicinity of a bottom surface X″ of the measurement container 1.

The change in the density of cells in the measurement solution X per unit time becomes maximum at the liquid surface X′ of the measurement solution X and at the bottom surface X″. However, at the bottom surface X″ of the measurement container 1, the cell density increases as time proceeds. Therefore, a larger ΔAbs can be obtained by measuring the absorbance at the liquid surface' of the measurement solution X or at the bottom surface X″, and the temporal change in the density of cells can be accurately detected.

### Second Embodiment

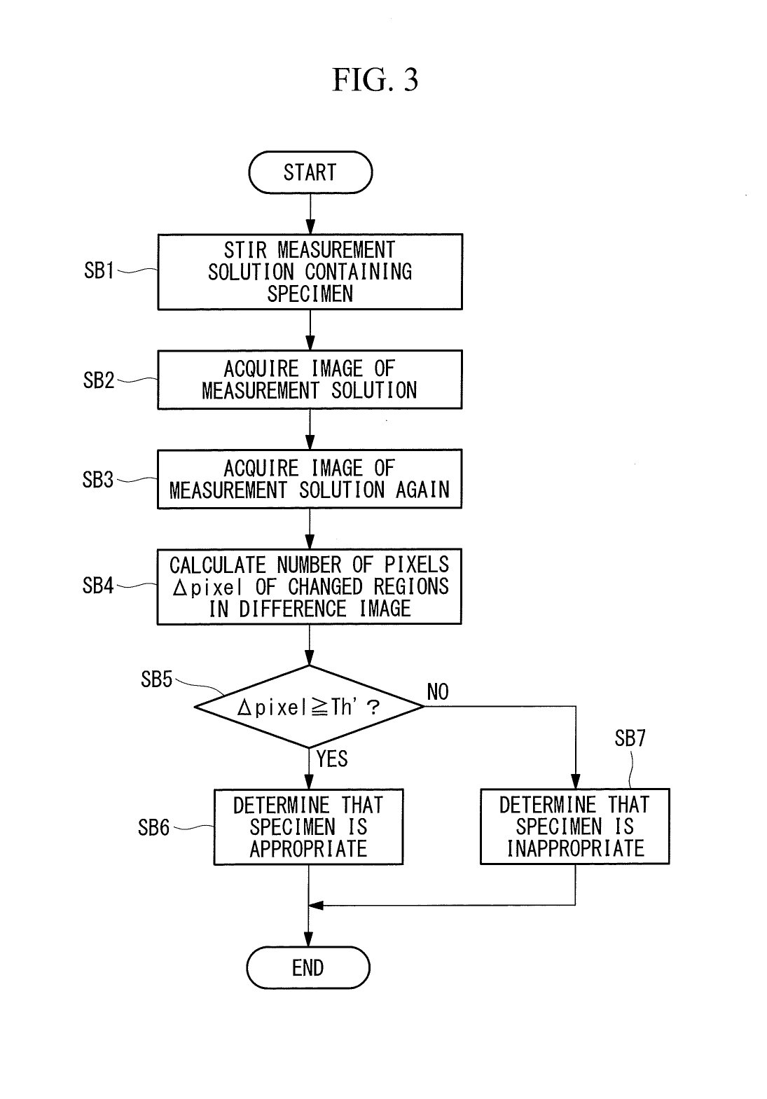

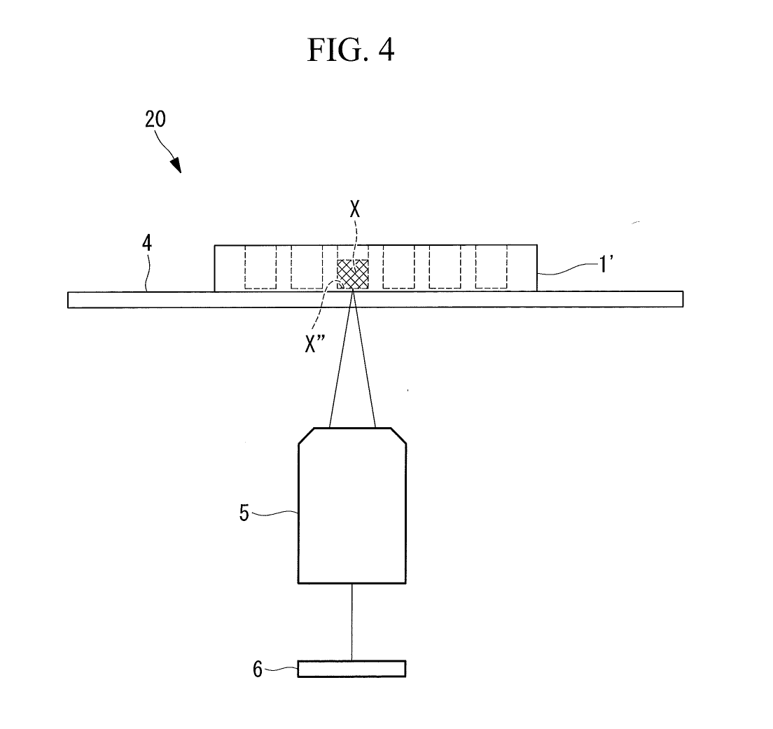

Next, a specimen evaluation method according to a second embodiment of the present invention will be described with reference to FIGS. 3 and 4.

As shown in FIG. 3, the specimen evaluation method of this embodiment includes: a stirring step SB1; an image-acquisition step (measurement step) SB2 of acquiring an image of the measurement solution; a re-acquisition step (remeasurement step) SB3 of acquiring again an image of the measurement solution after a time interval since the image-acquisition step SB2; a change calculation step SB4 of generating a difference image between the two images acquired in the image-acquisition step SB2 and the re-acquisition step SB3; and an evaluation steps SB5, SB6, and SB7 of evaluating whether or not the specimen is appropriate for examination on the basis of the obtained difference image.

The stirring step SB1 is the same as the stirring step SA1, which is described in the first embodiment.

In the image-acquisition step SB2, an image of the measurement solution X at the bottom surface X″ of a measurement container 1′ is acquired by using a general bright field microscope 20, as shown in FIG. 4. The bright field microscope 20 is provided with: a stage 4 on which the measurement container 1′ is placed; an objective lens 5 for observing a sample on the stage 4; and an imaging device 6 that acquires an image of the sample obtained by the objective lens 5. It is preferred that the magnification of the objective lens 5 be about 2 to 20 times. As the measurement container 1′, a measurement container that is made of an optically transparent material, such as a microplate generally used in cell measurement, is used.

Next, in the re-acquisition step SB3, an image of the measurement solution X at the bottom surface X″ of the measurement container 1′, the image having the same image-acquisition range as in the image-acquisition step SB2, is acquired. The cells in the measurement solution X settle out on the bottom surface X″ as time proceeds, and thus, the second image, which is acquired in the re-acquisition step SB3, includes more cell images than the first image, which is acquired in the image-acquisition step SB2.

The next change calculation step SB4 and the evaluation steps SB5, SB6, and SB7 are performed, for example, in an information-processing device, such as a computer, connected to the bright field microscope 20, by receiving the acquired first image and second image from the bright field microscope 20 and by processing the two images by using image processing software installed therein.

In the change calculation step SB4, first, a difference image between the first image, which is acquired in the image-acquisition step SB2, and the second image, which is acquired in the re-acquisition step SB3, is generated. Specifically, the difference image is generated by calculating the differences between the pixel values of pixels of the first image and the pixel values of the pixels of the second image and by setting the absolute values of the calculated differences as the pixel values at those pixels. Accordingly, changed regions that change between the first image and the second image, i.e., cells that exist in the second image but do not exist in the first image, are extracted. In the difference image, the extracted changed regions are displayed as bright regions having large pixel values, and the other regions (hereinafter, referred to as “unchanged regions”) are displayed as dark regions having almost-zero pixel values.

Next, the number of pixels Δpixel (difference; hereinafter, simply referred to as “Δpixel”) constituting the changed regions is calculated. The difference Δpixel corresponds to the temporal change in the density of cells at the bottom surface X″ of the measurement container 1′ and is proportional to the total number of cells contained in the entire measurement solution X. In order to more clearly distinguish between the changed regions and the unchanged regions, the difference image may be digitalized to display the changed regions in white or black and the unchanged regions in black or white.

In the evaluation steps SB5, SB6, and SB7, Δpixel obtained in the change calculation step SB4 is compared with a predetermined threshold Th′ (Step SB5). Then, if Δpixel is equal to or larger than the predetermined threshold Th′ (YES in Step SB5), it is determined that the number of cells necessary for examination is contained in the specimen and that this specimen is thus appropriate for examination (Step SB6). On the other hand, if Δpixel is smaller than the predetermined threshold Th′ (NO in Step SB5), it is determined that the number of cells contained in the specimen is not sufficient for the examination and that this specimen is thus not appropriate for examination (Step SB7). Here, the predetermined threshold Th′ is set on the basis of the number of cells necessary for examination for which the specimen is provided. The examination may be pathological examination, in which tissue diagnosis and cytology are performed, may be measurement of a target protein, which is performed by a colorimetric method or immunoenzymetric assay, or may be a genetic test.

Here, the images acquired in the image-acquisition step SB2 and the re-acquisition step SB3 each include, in addition to cells, other biological materials. However, as described above, because the settling velocities of the other biological materials are quite small, a change between the first image and the second image that is derived from the biological materials is vanishingly small. Therefore, Δpixel obtained from the difference image corresponds to the temporal change in the density of the net cells. By calculating this difference Δpixel, it is possible to estimate an accurate number of cells contained in the specimen and to accurately evaluate whether or not the specimen is appropriate for examination.

### EXAMPLES

### Example 1

Next, Example 1 of the specimen evaluation method according to the above-described first embodiment will be described.

In this example, a specimen collected through FNA was used. In FNA, a 23-gauge intravenous needle and a syringe that were connected to each other by using a three-way stopcock were used. Specifically, a chicken liver was punctured with the intravenous needle, and a specimen was collected through suction by using the syringe.

The collected specimen was added to a normal saline solution (measurement solution), and the normal saline solution was sufficiently stirred, thereby preparing a sample A. Next, 300 μL of the sample A was dispensed to a 96-well plate (manufactured by Becton, Dickinson and Company, catalog No. 351172).

Next, the absorbance at an upper layer portion of the sample A at each of wavelengths 400 nm, 600 nm, and 800 nm was measured by using a plate reader (CORONA ELECTRIC Co. Ltd., SH-8100) (the measurement step, the remeasurement step). Here, the sample A in the wells was stirred for five seconds through pipetting (the stirring step), a first absorbance measurement was performed immediately after the completion of the stirring (0 seconds) (the measurement step), and, after that, the measurements were performed at 10-second intervals up to 180 seconds (the remeasurement step). Then, the difference ΔAbs in absorbance at 0 seconds and 180 seconds was calculated (the change calculation step).

A comparative sample that was obtained by diluting chicken whole blood (Cosmo Bio, 12075505) tenfold with a normal saline solution and that did not include cells was also prepared. The absorbance of the comparative sample was also measured by the same method used for the sample A.

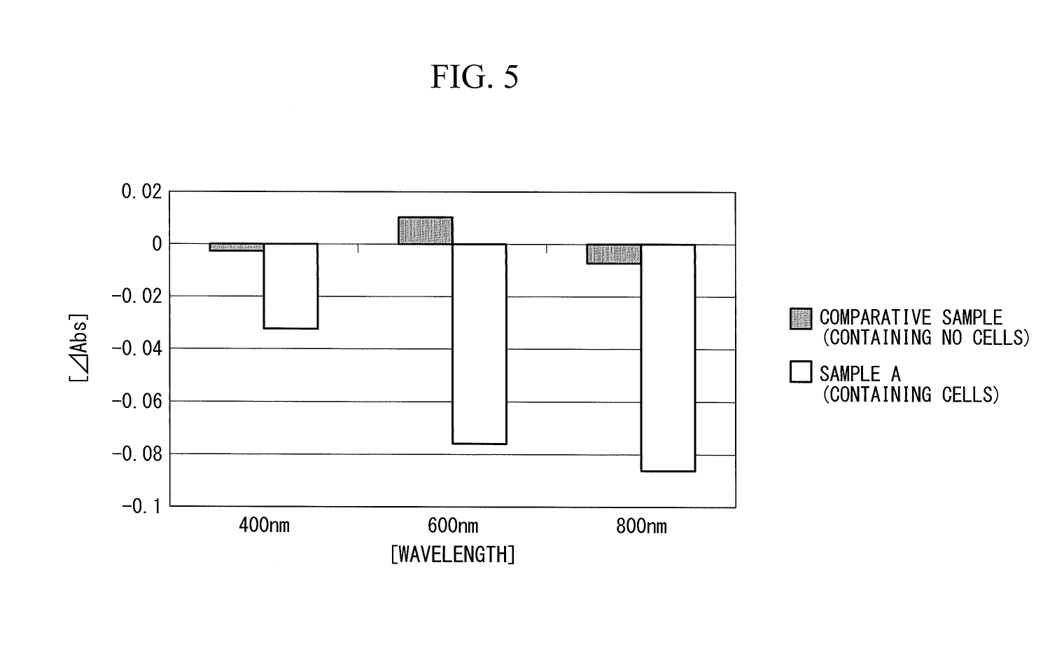

FIG. 5 shows ΔAbs of the sample A and the comparative sample at the respective measurement wavelengths. In the sample A, obvious reductions in absorbance were confirmed at all the measurement wavelengths 400, 600, and 800 nm. On the other hand, in the comparative sample, which did not include cells, significant reductions in absorbance were not found. From these results, it was confirmed that the reductions in the absorbance of the sample A were derived from the cells and that the difference ΔAbs, which correlated with the presence or absence of a cell and the number of cells, could be obtained from the sample A, which contained blood components.

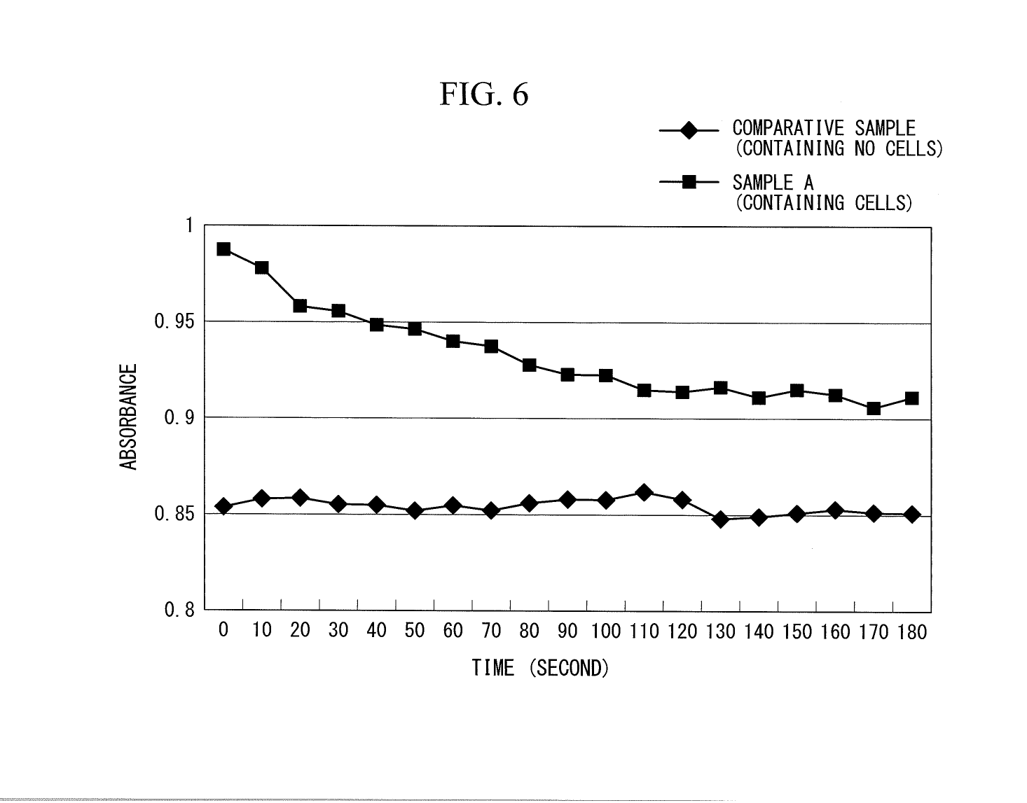

FIG. 6 shows the temporal changes in the absorbances of the sample A and the comparative sample, at a representative wavelength of 600 nm, from 0 to 180 seconds. In FIG. 6, the absorbance of the sample A decreased immediately after the completion of stirring and remained unchanged after about 120 seconds. On the other hand, in the comparative sample, a significant temporal change in the absorbance was not recognized. In this Example, the absorbances at the wavelength of 600 nm, for example, were measured at 0 seconds and 60 seconds, and the predetermined threshold Th for ΔAbs was set to 0.04, thereby making it possible to evaluate whether or not a sufficient number of cells was contained in the specimen.

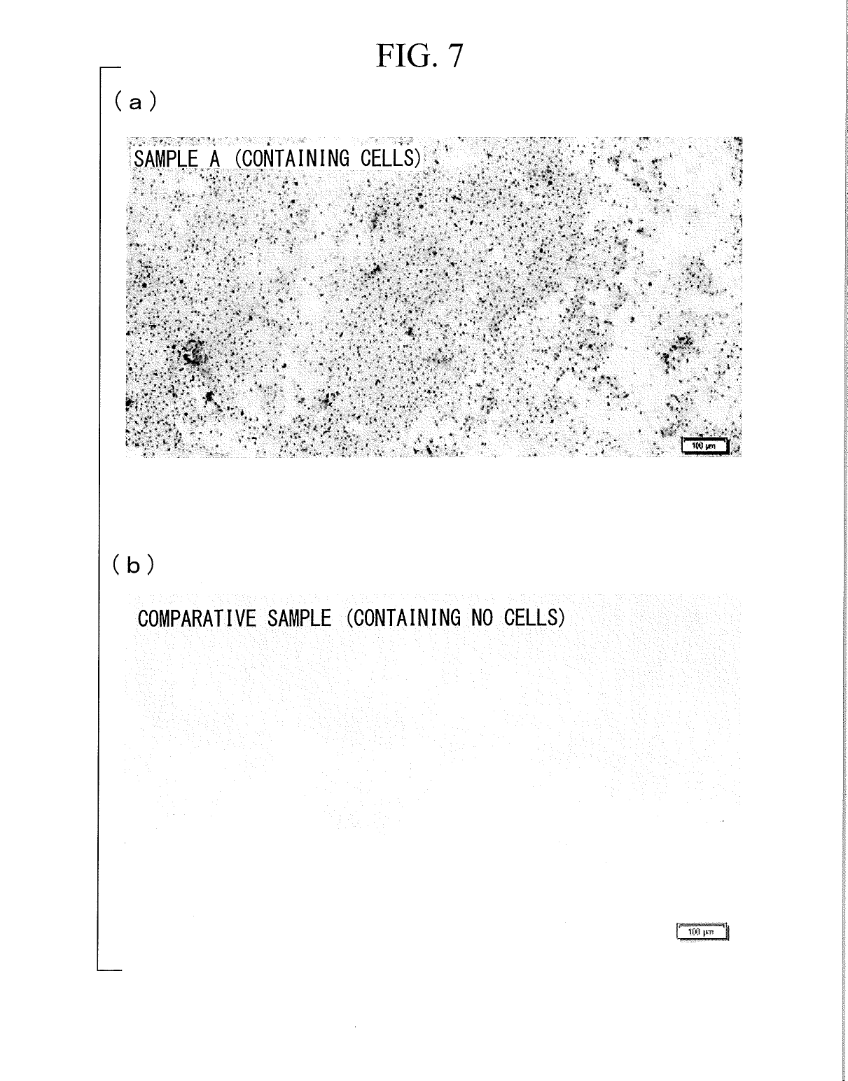

FIGS. 7(a) and 7(b) show images of slide samples generated by dropping the sample A and the comparative sample on slide glasses and of samples in which cells were subjected to Papanicolaou staining. As shown in FIG. 7(a), many cells were observed in the slide sample of the sample A. On the other hand, as shown in FIG. 7(b), no cells at all were found in the slide sample of the comparative sample.

### Example 2

Next, Example 2 of the specimen evaluation method according to the above-described second embodiment will be described.

In this Example, samples A, B, C, D, and E were used. The sample A was the same as the sample A used in Example 1. The sample B was prepared in the same way as the sample A except that a specimen that was collected by using a 26-gauge intravenous needle, instead of the 23-gauge intravenous needle, was used. The sample C was a cell suspension in which cultured cells (A549 cell line) were suspended in a normal saline solution and was prepared so as to have a cell density of 1.33×10⁵cells/mL. The sample D was prepared by diluting the cell suspension of the sample C tenfold (1.33×10⁴ cells/mL). The sample E was prepared by diluting the cell suspension of the sample C one-hundredfold (1.33×10³ cells/mL). In this Example, a normal saline solution was used as a comparative sample.

300 μL of each of the samples A, B, C, D, and E was dispensed to a 96-well microplate (manufactured by Becton, Dickinson and Company, catalog No. 351172). Next, while observing the bottom surface of the microplate accommodating the sample A, B, C, D, or E with an inverted optical microscope (manufactured by Olympus Corporation, CK X41), the sample A, B, C, D, or E in the wells was stirred by using a micropipette (the stirring step), first image-acquisition was performed immediately after the completion of the stirring (0 seconds), by using a digital camera (manufactured by Olympus Corporation, DP21) (the image-acquisition step), and image-acquistion was performed again after 60 seconds (the re-acquisition step). Images of the sample A were additionally acquired after 10 seconds and 30 seconds as well.

Next, image-processing software was used to generate, from two images acquired at 0 seconds and 60 seconds, a difference image that was digitalized such that a changed region was displayed in white, and an unchanged region was displayed in black. Next, the number of pixels Δpixel constituting white regions was obtained from a histogram of pixel values of the difference image (the change calculation step).

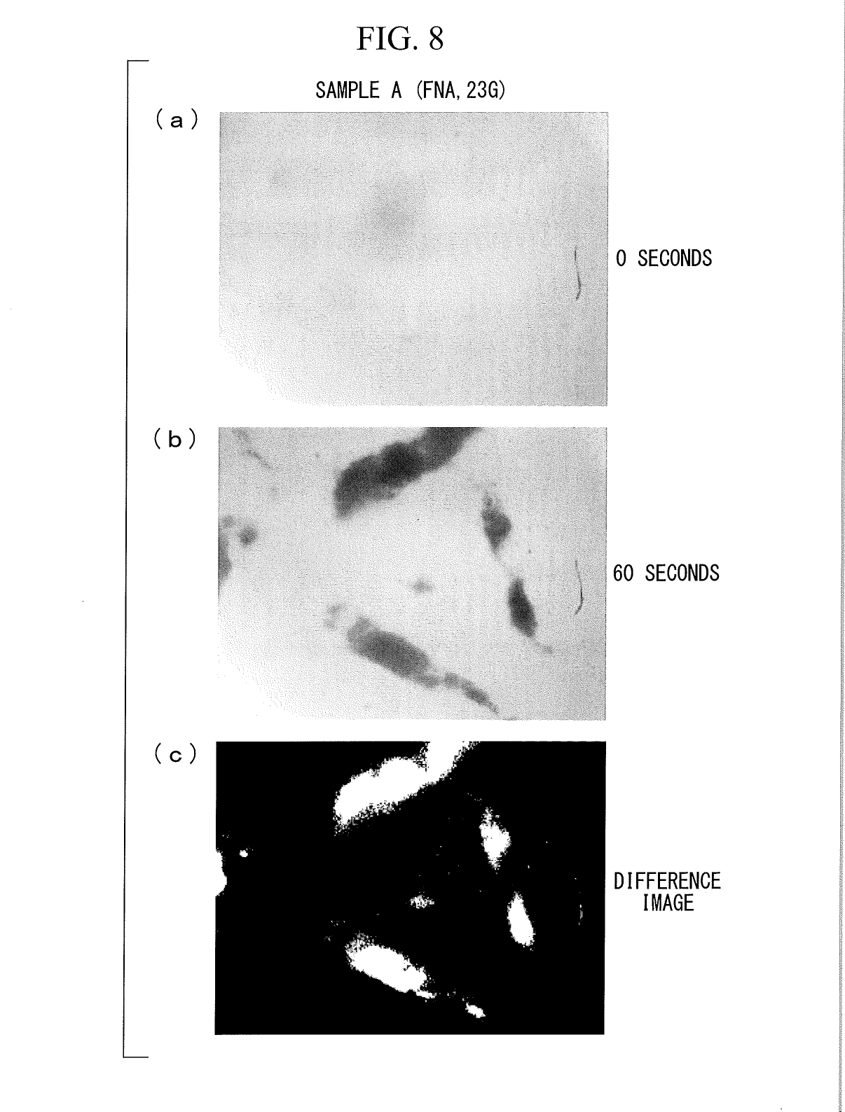

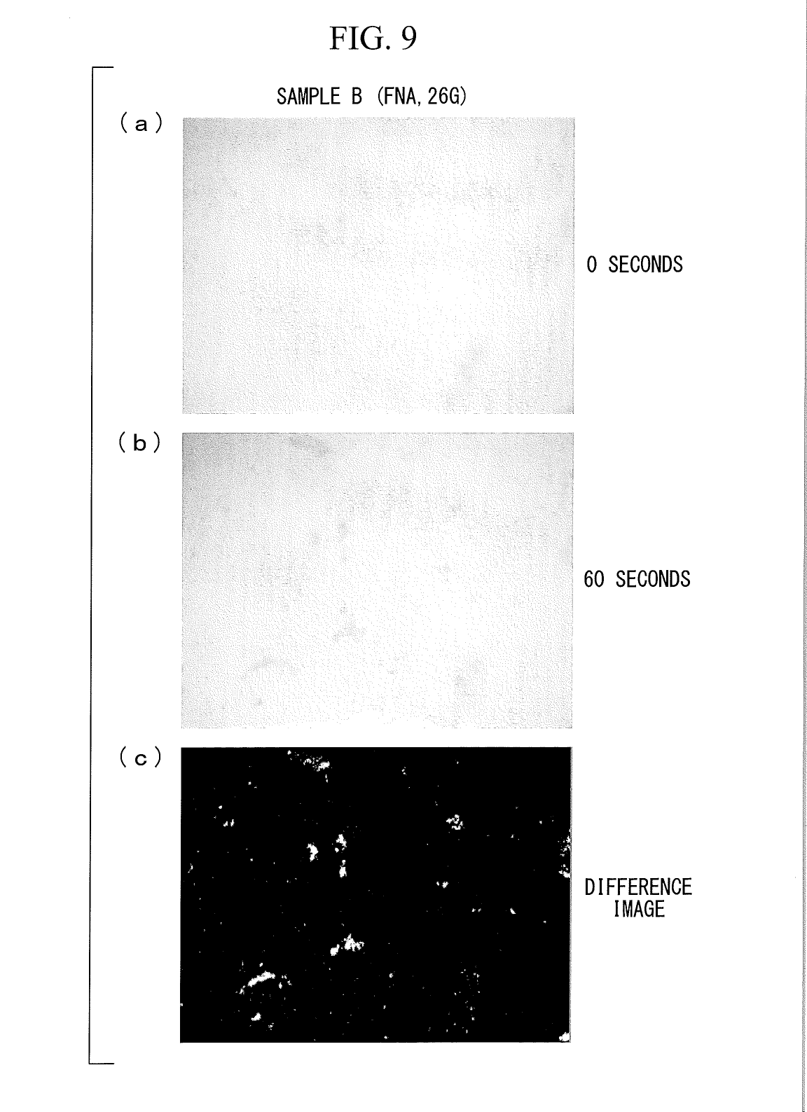

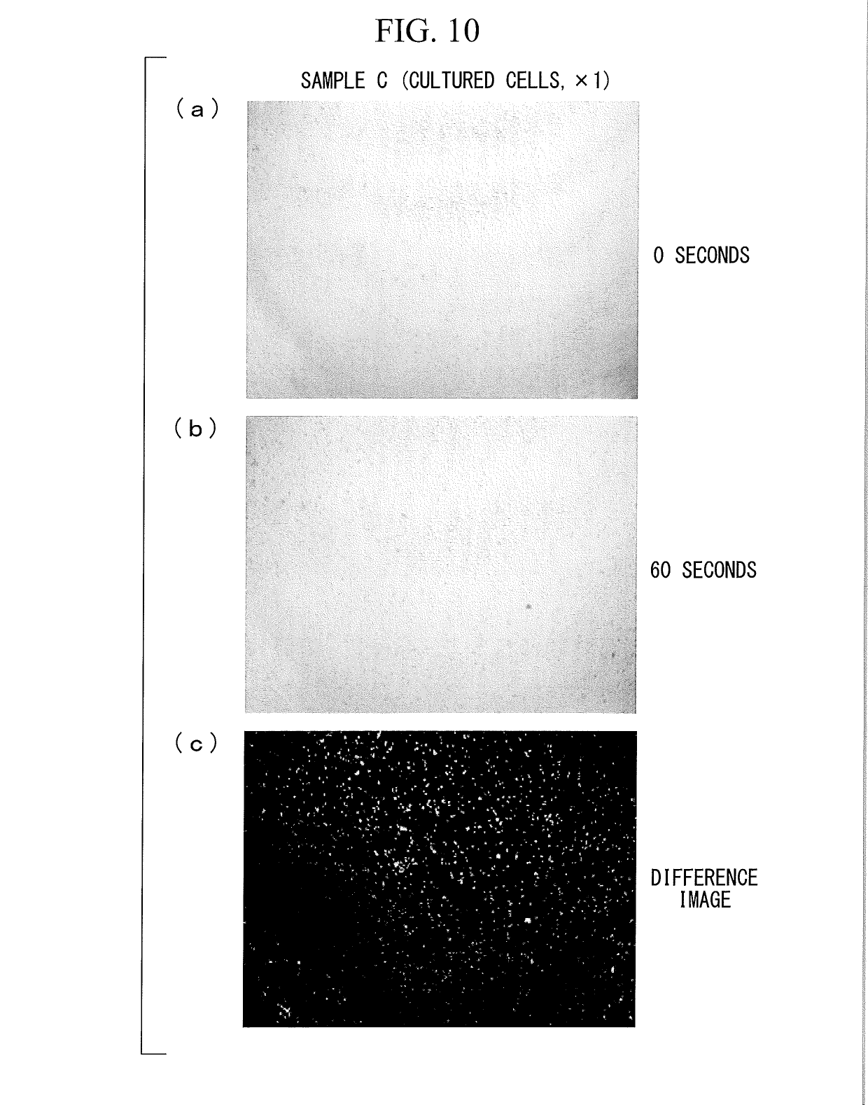

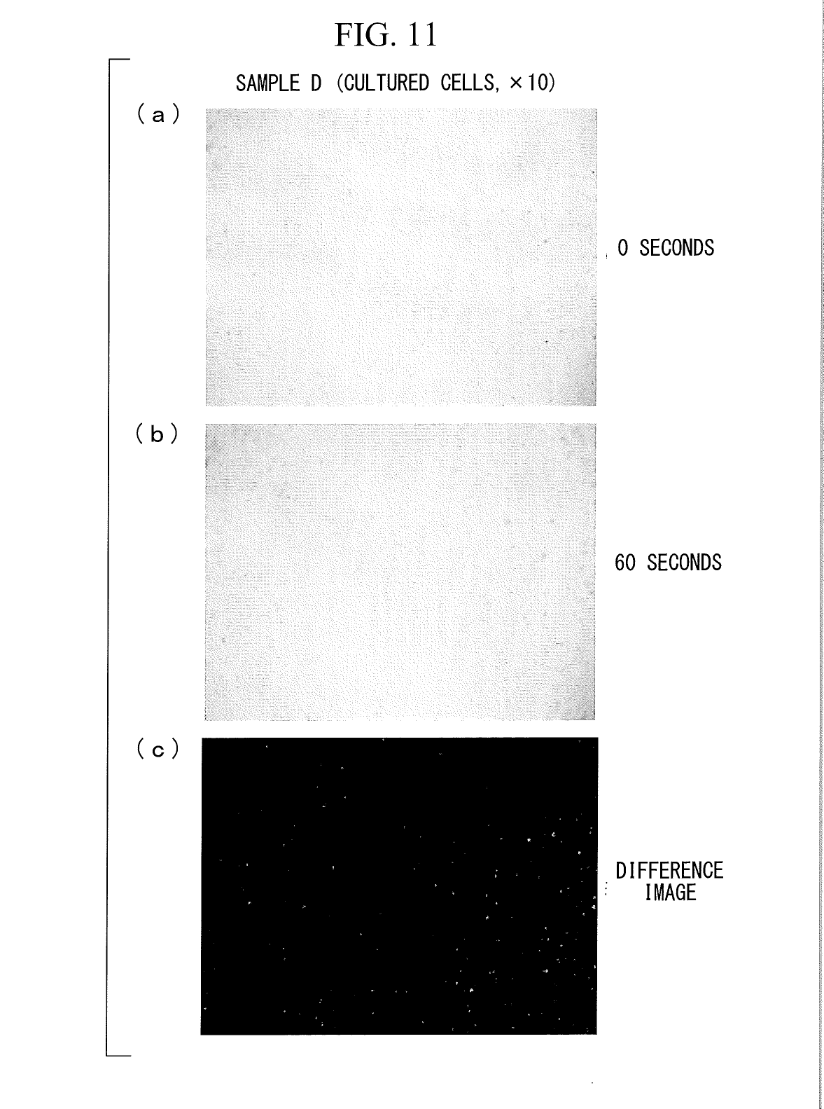

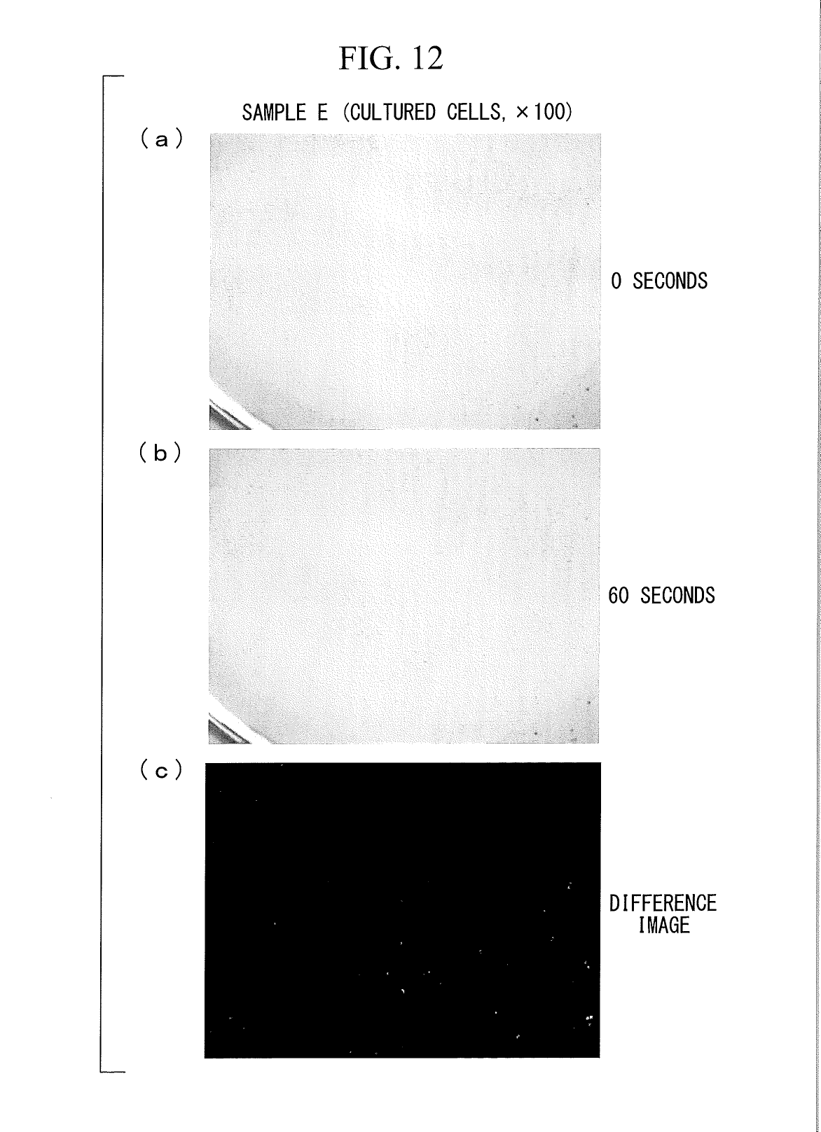

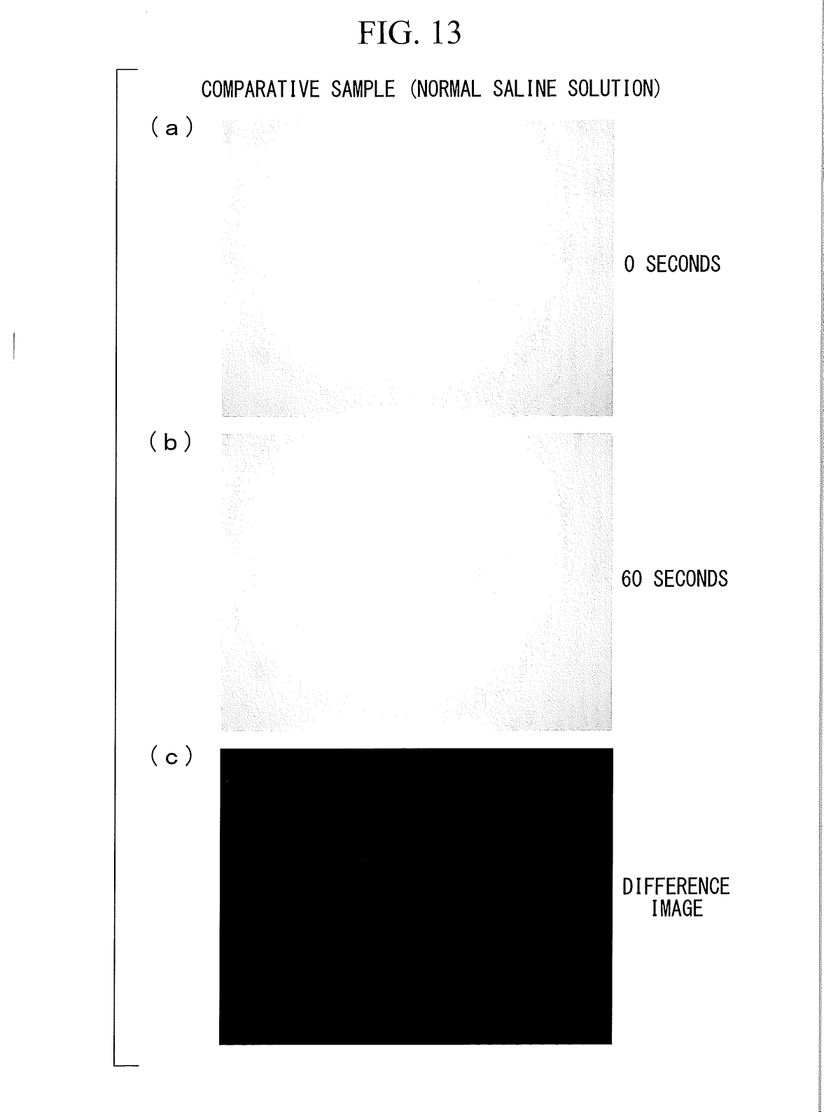

FIGS. 8 to 13 show images acquired at 0 seconds (see (a) in each figure) and 60 seconds (see (b) in each figure) and a difference image (see (c) in each figure), of the samples A, B, C, D, and E and the comparative sample. As shown in FIGS. 8 and 9, the sample A, in which a thick intravenous needle was used to collect the specimen, includes more cells than the sample B, in which a thin intravenous needle was used, and the total area of changed regions (white regions) in the difference image of the sample A was larger than that of the sample B. Furthermore, it was confirmed that the sample A includes a lot of blood because the background was red. As shown in FIGS. 10 to 12, as the number of cells contained in each of the samples C, D, and E was larger, the total area of changed regions in the difference image was larger. As shown in FIG. 13, the difference image of the comparative sample included almost no changed regions. From these results, it was confirmed that there was a positive correlation between the number of cells contained in each of the samples A, B, C, D, and E and Δpixel.

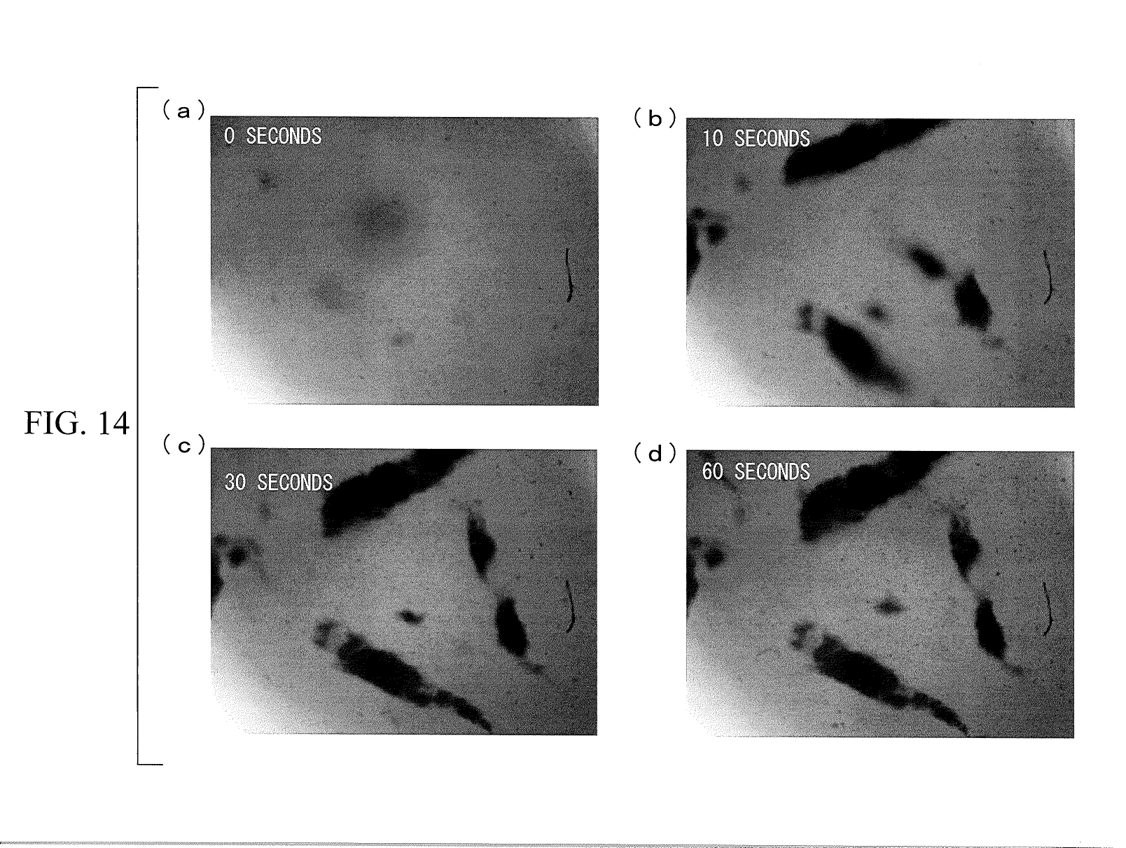

FIGS. 14(a) to 14(d) show images of the sample A acquired at 0 seconds, 10 seconds, 30 seconds, and 60 seconds. As shown in FIG. 14(b), settling of cell aggregates to the bottom surface of the measurement container was fast, and, after 10 seconds since the completion of stirring, many aggregates were already observed on the bottom surface.

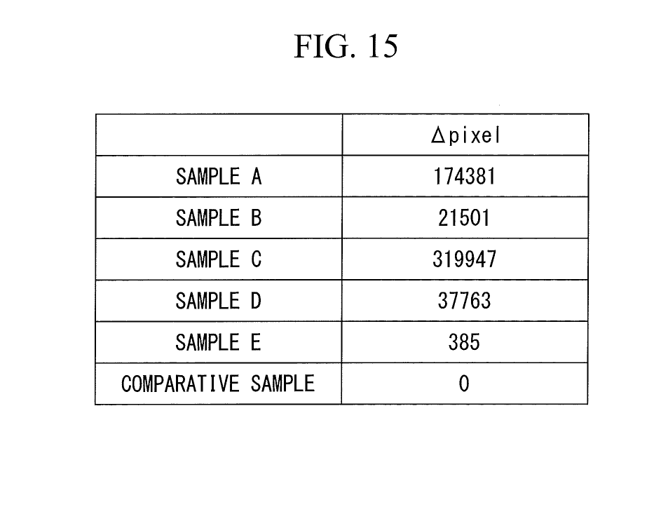

FIG. 15 shows Δpixel obtained from the difference images of the samples A, B, C, D, and E and the comparative sample.

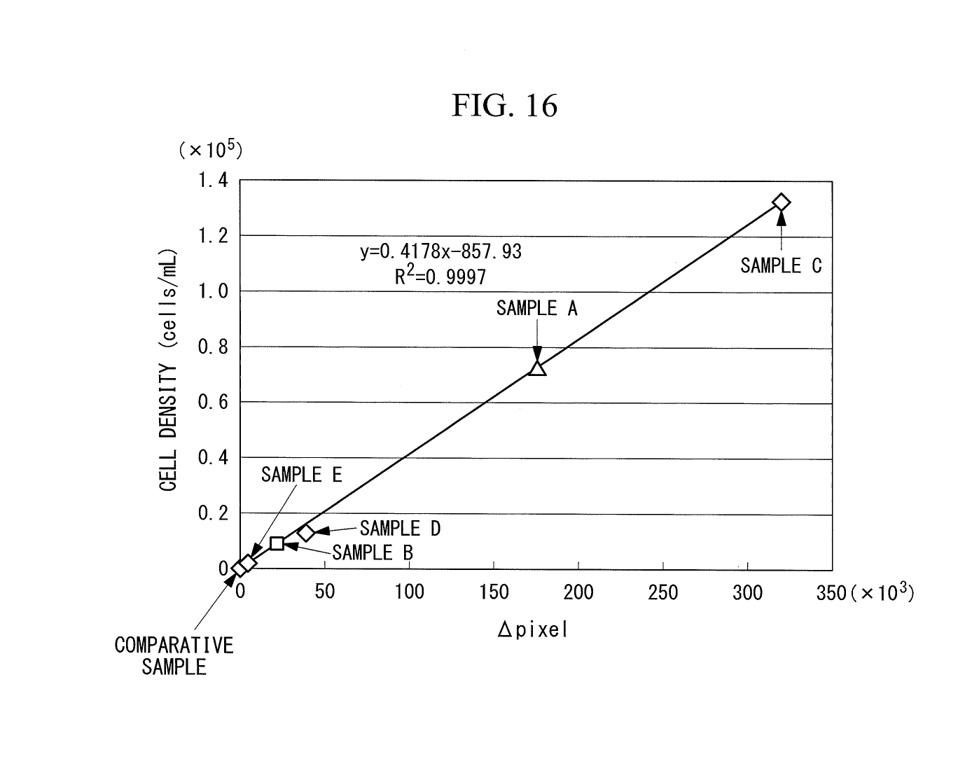

FIG. 16 is a graph in which the relationships between the cell density and Δpixel, of the samples C, D, and E and the comparative sample, whose cell densities were known, were plotted. As shown in FIG. 16, it was confirmed that Δpixel was proportional to the cell density. By using the graph of FIG. 16, it was possible to estimate that the cell density of the sample A was 71998 cells/mL, and the cell density of the sample B was 8551 cells/mL, from Δpixel of the samples A and B.

In this Example, for example, images were acquired at 0 seconds and 60 seconds after the completion of stirring, and the predetermined threshold Th′ for Δpixel was set to 14021 corresponding to a cell density of 5000 cells/mL, thereby making it possible to evaluate whether or not a sufficient number of cells was contained in the specimen.

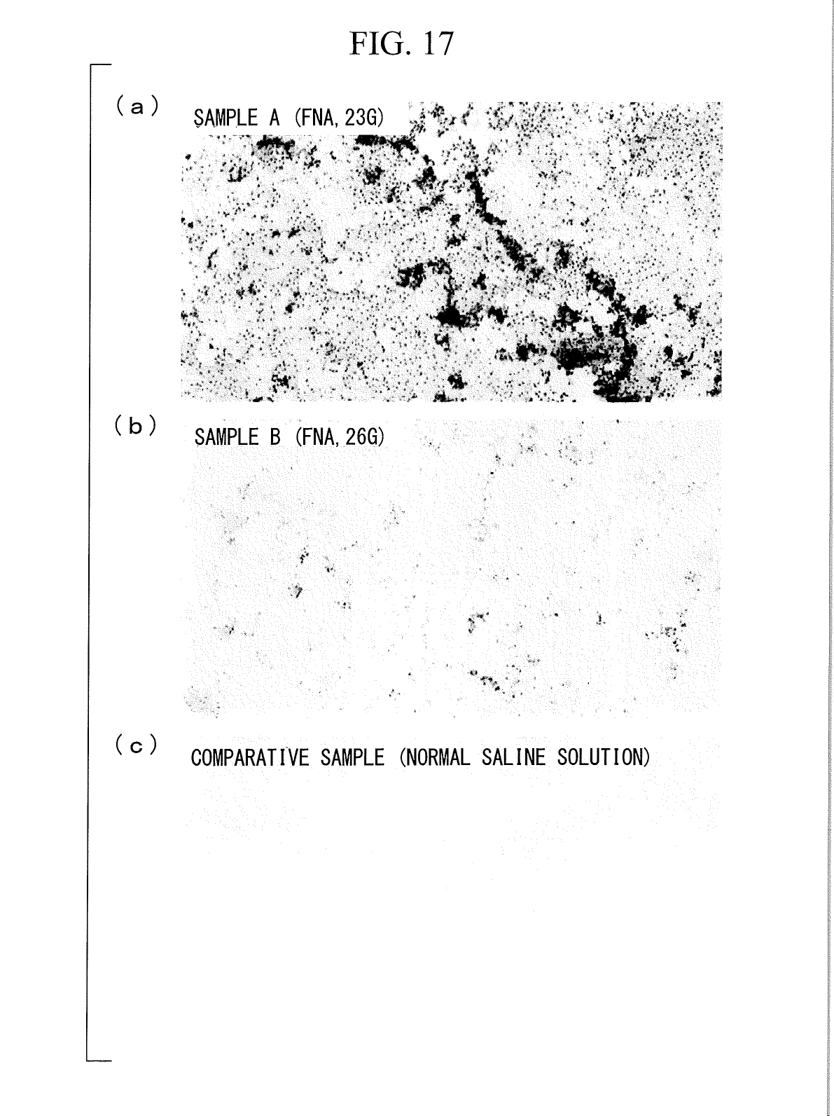

FIGS. 17(a), 17(b), and 17(c) show images of slide samples of the samples A and B and the comparative sample and of samples in which cells were subjected to Papanicolaou staining. As shown in FIGS. 17(a) and 17(b), many cells were observed in the slide samples of the samples A and B, and it was confirmed that the sample A contained more cells than the sample B. On the other hand, as shown in FIG. 17(c), no cells at all were found in the slide sample of the comparative sample.

The above-described embodiment leads to the following inventions.

The present invention provides a specimen evaluation method including: a stirring step of stirring a measurement solution that contains a specimen collected from a living body, thereby uniformly dispersing, in the measurement solution, cells contained in the specimen; a measurement step of measuring, after completion of the stirring in the stirring step, the density of the cells at a predetermined depth position in the measurement solution at a first time point; a remeasurement step of measuring again the density of the cells at the predetermined depth position in the measurement solution at a second time point after a time interval since the first time point; a change calculation step of calculating the difference between the density of the cells measured in the measurement step and the density of the cells measured in the remeasurement step; and an evaluation step of evaluating whether the specimen is good or not on the basis of the difference in the density of the cells calculated in the change calculation step.

According to the present invention, the density of the cells at the same depth position in the measurement solution stirred in the stirring step is measured twice in the measurement step and the remeasurement step with a time interval therebetween. Because the cells in the measurement solution settle as time proceeds, the density of the cells at each depth position in the measurement solution changes over time. Therefore, different measured values of the density of the cells are obtained in the measurement step and the remeasurement step. Here, when the specimen is a mixed sample containing, in addition to cells, other biological materials, the measured values obtained in the measurement step and the remeasurement step each include a background value that is derived from other materials in addition to the measured value of the density of the net cells.

Cells are much heavier than other biological materials, and the settling velocities of other biological materials are vanishingly quite small in comparison to the settling velocity of the cells. Specifically, the background values included in the two measured values obtained in the two measurements are equal to each other. Therefore, by calculating the difference between the two measured values in the change calculation step, it is possible to detect an accurate temporal change in the density of the cells from the first time point to the second time point, from which the influence of the background value is removed. The temporal change in the density of the cells is correlated with the number of cells contained in the specimen. In the evaluation step, it is possible to estimate an accurate number of cells contained in the specimen from the difference between the measured values, which is obtained in the change calculation step, and to accurately evaluate whether or not the specimen is appropriate for examination on the basis of the estimated number of cells.

In the above-described invention, in the measurement step and the remeasurement step, turbidity may be measured at the predetermined depth position in the measurement solution; and, in the change calculation step, the difference between the turbidity obtained in the measurement step and the turbidity obtained in the remeasurement step may be calculated.

By doing so, the turbidity of the measurement solution is proportional to the density of the cells in the measurement solution; therefore, the density of the cells can be easily measured in a non-contact manner, on the basis of the turbidity.

In the above-described invention, in the measurement step and the remeasurement step, images at the predetermined depth position in the measurement solution may be acquired; and, in the change calculation step, the area of a region that changes between the image acquired in the measurement step and the image acquired in the remeasurement step may be calculated.

By doing so, because the images of the measurement solution include the images of the cells in the measurement solution, the density of the cells can be easily measured in a non-contact manner, on the basis of the numbers of cells in the images.

In the above-described invention, it is preferred that the predetermined depth position be a bottom surface of the measurement solution accommodated in a measurement container or in the vicinity of the bottom surface or be a liquid surface thereof or in the vicinity of the liquid surface.

By doing so, it is possible to detect the temporal change in the density of the cells with high accuracy by measuring the density of the cells at the bottom surface or at the liquid surface, where the change in the density of the cells per unit time becomes maximum, or in the vicinity of the bottom surface or the liquid surface.

In the above-described invention, it is preferred that the first time point be immediately after completion of the stirring in the stirring step, and it is preferred that the second time point fall within 120 seconds from completion of the stirring in the stirring step.

Settling of the cells in the measurement solution starts immediately after completion of the stirring of the measurement solution, and the time rate of change in the density of the cells tends to decrease as time proceeds. Therefore, a first measurement is performed immediately after completion of the stirring, and a second measurement is performed within 120 seconds after completion of the stirring, thereby making it possible to detect the temporal change in the density of the cells with high accuracy.

In the above-described invention, it is preferred that the time interval between the first time point and the second time point fall within a range of 10 seconds to 120 seconds, both inclusive.

By doing so, it is possible to realize both detection accuracy of the temporal change in the density of the cells and work efficiency. If the time interval between the two measurements is shorter than 10 seconds, there is a possibility that the temporal change in the density of the cells is too small to accurately evaluate the specimen. On the other hand, even if the time interval between the two measurements is made longer than 120 seconds, a further increase in the obtained temporal change in the density of the cells cannot be expected.

### REFERENCE SIGNS LIST

1 measurement container
2 light source
3 detector
4 stage
5 objective lens
6 imaging device
10 spectrophotometer
20 bright field microscope
X measurement solution
SA1, SB1 stirring step
SA2 measurement step
SA3 remeasurement step
SB2 image-acquisition step (measurement step)
SB3 -acquisition step (remeasurement step)
SA4, SB4 change calculation step
SA5, SA6, SA7, SB5, SB6, SB7 evaluation step

1. A specimen evaluation method comprising:
a stirring step of stirring a measurement solution that contains a specimen collected from a living body, thereby uniformly dispersing, in the measurement solution, cells contained in the specimen;
a measurement step of measuring, after completion of the stirring in the stirring step, the density of the cells at a predetermined depth position in the measurement solution at a first time point;
a remeasurement step of measuring again the density of the cells at the predetermined depth position in the measurement solution at a second time point after a time interval since the first time point;
a change calculation step of calculating the difference between the density of the cells measured in the measurement step and the density of the cells measured in the remeasurement step; and
an evaluation step of evaluating whether the specimen is good or not on the basis of the difference in the density of the cells calculated in the change calculation step.

2. A specimen evaluation method according to claim 1,
wherein, in the measurement step and the remeasurement step, turbidity is measured at the predetermined depth position in the measurement solution; and
in the change calculation step, the difference between the turbidity obtained in the measurement step and the turbidity obtained in the remeasurement step is calculated.

3. A specimen evaluation method according to claim 1,
wherein, in the measurement step and the remeasurement step, images at the predetermined depth position in the measurement solution are acquired; and
in the change calculation step, the area of a region that changes between the image acquired in the measurement step and the image acquired in the remeasurement step is calculated.

4. A specimen evaluation method according to claim 1, wherein the predetermined depth position is a bottom surface of the measurement solution accommodated in a measurement container or in the vicinity of the bottom surface or is a liquid surface thereof or in the vicinity of the liquid surface.

5. A specimen evalution method according to claim 1, wherein the first time point is immediately after completion of the stirring in the stirring step.

6. A specimen evaluation method according to claim 1, wherein the second time point falls within 120 seconds from completion of the stirring in the stirring step.

7. A specimen evaluation method according to claims 1, wherein the time interval between the first time point and the second time point falls within a range of 10 seconds to 120 seconds, both inclusive.
